(19) United States
(12) Patent Application Publication
(10) Pub. No.: US 2006/0178519 A1
(43) Pub. Date: Aug. 10, 2006

(54) Process for preparing tegaserod

(76) Inventors: Venkataraman Sundaram, Hyderabad (IN); Srinivasulu Gudipati, Hyderabad (IN); Venkata Naga Brahmeswararao Mandava, Hyderabad (IN); Goverdhan Banda Reddy, Hyderabad (IN); Radhakrishna Singamsetty, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/315,859

(22) Filed: Dec. 22, 2005

Related U.S. Application Data
(60) Provisional application No. 60/639,159, filed on Dec. 23, 2004.

(51) Int. Cl. C07D 209/14

(52) U.S. Cl. 548/505

(57) ABSTRACT

A process for preparing tegaserod.

### CROSS-REFERENCE TO RELATED APPLICATION

The present application is a nonprovisional filing of copending U.S. Provisional Application No. 60/639,159 filed on Dec. 23, 2004, the entire content of which is incorporated herein by this reference.

### INTRODUCTION TO THE INVENTION

The present invention relates to an improved process for preparing substantially pure tegaserod and its pharmaceutically acceptable salts, in high yields.

Tegaserod is chemically known as 3-(5-methoxy-1H-indol-3-ylmethylene)-N-pentylcarbazimidamide and can be depicted structurally by Formula I.

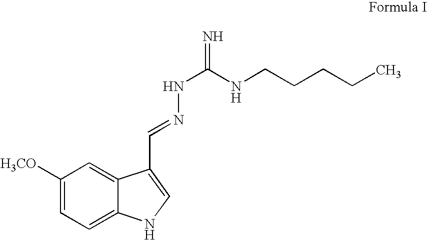

Tegaserod is the first and only medicine proven to relieve all three symptoms of irritable bowel syndrome with constipation in women viz. Abdominal pain or discomfort, Bloating, and Constipation—the “ABCs.” Tegaserod has a different mechanism of action when compared with other medications also useful in such conditions such as laxatives, fibers, and other medications. It blocks the 5-HT4 receptor and prevents seretonin from binding to it resulting in increased contractions. Tegaserod maleate is available commercially under the trade name “ZELNORM” as white, round tablets in 2 and 6 mg strengths.

Tegaserod maleate was disclosed in U.S. Pat. No. 5,510,353. The patent also discloses the preparation of tegaserod by reacting aminoguanidine hydrochloride of Formula II with n-pentylamine of Formula III to give N-pentyl-N-aminoguanidine hydrochloride of Formula IV, which in turn is condensed with 5-methoxy-1H-indole-3 carboxaldehyde of Formula V in a protic solvent in the presence of an inorganic or organic acid to give tegaserod base. The whole process can be depicted as given in Scheme 1. 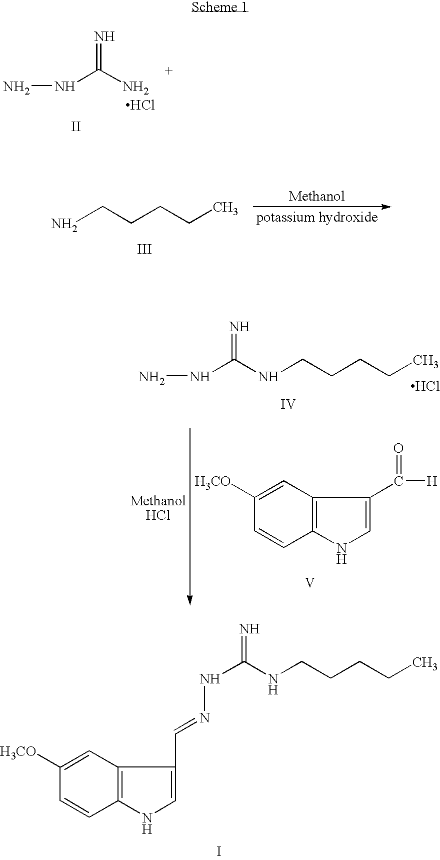
The above-mentioned process involves isolation of intermediates at all stages leading to lower yields and a longer time cycle. Hence a process involving a one-pot synthesis may help in overcoming the problems in the prior art process.

The above process uses methanol and discloses the use of lower alcohols for the condensation of N-pentyl-N-aminoguanidine hydrochloride of Formula IV with 5-methoxy-1H-indole-3 carboxaldehyde of Formula V. It has been found that the product has a high solubility in methanol. When reactions are conducted in methanol, there is a considerable amount of yield loss during the workup. Hence, a change in the solvent for the above condensation reaction will be helpful in producing better yields.

The present invention provides an improved process for the preparation of substantially pure tegaserod and its pharmaceutically acceptable salts in high yields, which is safe, cost effective, and industrially feasible.

### SUMMARY OF THE INVENTION

The present invention relates to an improved process for preparing substantially pure tegaserod and its pharmaceutically acceptable salts in high yields.

The invention provides a process for the preparation of substantially pure tegaserod and its pharmaceutically acceptable salts in high yields comprising the steps of:

a) reacting S-methylthiosemicarbazide hydroiodide of Formula VI with n-pentylamine of Formula III in the presence of a suitable solvent to give N-pentyl-N-aminoguanidine hydroiodide of Formula VII, which may or may not be isolated;

b) condensation of the N-pentyl-N′-aminoguanidine hydroiodide of Formula VII with 5-methoxy-1H-indole-3-carboxaldehyde of Formula V in the presence of a suitable solvent in an acidic medium to give 3-(5-methoxy-1H-indol-3-ylmethylene)-N-pentylcarbazimidamide (tegaserod free base) of Formula I.

According to an embodiment of the invention, the intermediate compound formed in step a) is not isolated, but is carried through to conversion in situ into tegaserod of Formula I.

In another aspect, the invention provides substantially pure tegaserod containing very low concentrations of any one or more of impurities, including
a) [(5-methoxy-1H-indol-3-yl)methylene-amino] guanidine HCl of Formula IX (hereinafter referred to as the “aminoguanidine impurity”); and
b) (5-methoxy-1H-indol-3-yl) methylene-amino]-2-methyl isothiourea hydroiodide of Formula X (hereinafter referred to as the “S-methyl impurity”).

In a further aspect of the invention, there is provided a process for the preparation of the aminoguanidine impurity of Formula IX, and the S-methyl impurity of Formula X.

### DETAILED DESCRIPTION

The present invention in one aspect relates to substantially pure tegaserod and its pharmaceutically acceptable salts.

In another aspect, the invention provides a process for the preparation of substantially pure tegaserod and its maleate salt in high yields comprising the steps of:

a) reacting S-methylthiosemicarbazide hydroiodide of Formula VI with n-pentylamine of Formula III in the presence of a suitable solvent to give N-pentyl-N-aminoguanidine hydroiodide of Formula VII, which may or may not be isolated; and

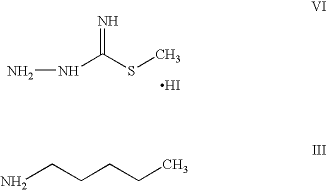

b) condensing the product of step a) with 5-methoxy-1H-indole-3-carboxaldehyde of Formula V in the presence of a suitable solvent, and in an acidic medium, to give 3-(5-methoxy-1H-indol-3-ylmethylene)-N-pentylcarbazimidamide of Formula I.

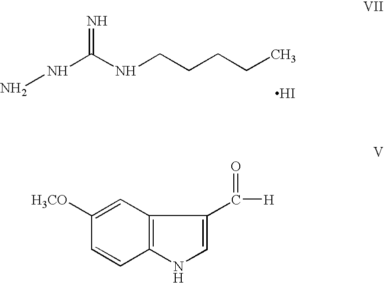

The product of step b), which is the tegaserod compound of Formula I, can be purified by slurrying, recrystallization, or a combination thereof in a suitable solvent.

Tegaserod free base of Formula I can be converted into its maleate salt of Formula VIII by reacting with maleic acid.

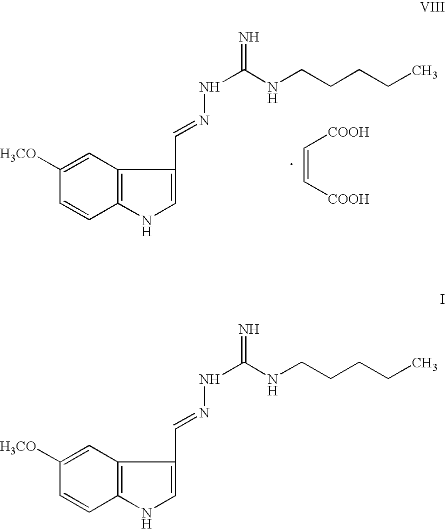

The whole process can be depicted as given in Scheme 2.

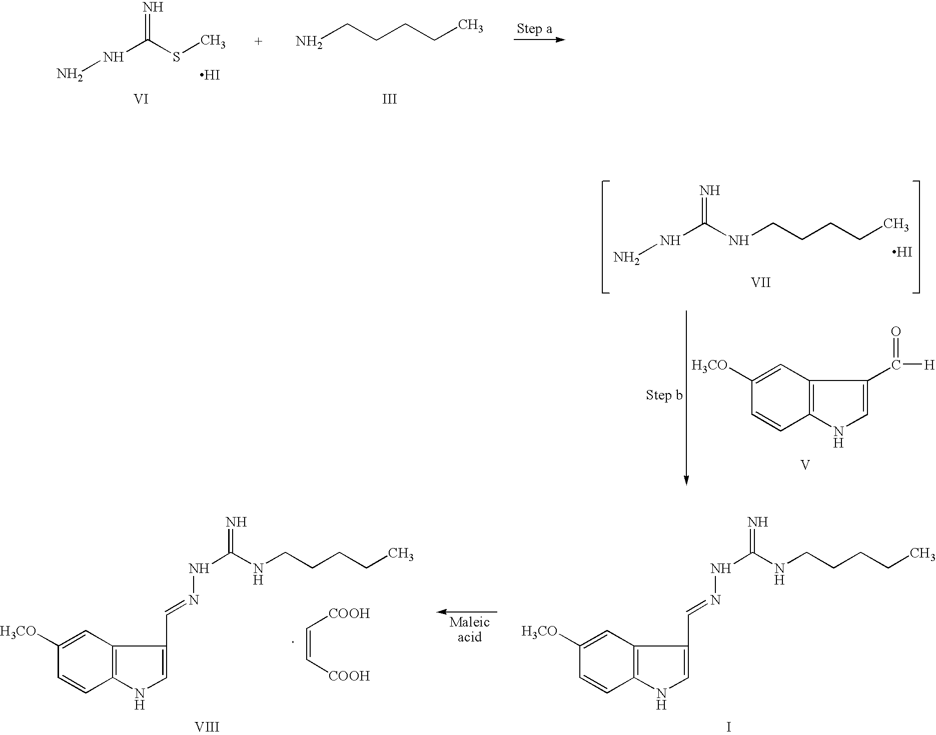

According to an important embodiment of the invention, the step a) can be carried out without isolating the intermediate compound such as in step a) wherein the product is carried further without isolation and is then converted in situ into tegaserod of Formula I according to step b).

Accordingly, S-methylthiosemicarbazide hydroiodide of Formula VI is reacted with n-pentylamine of Formula III in the presence of a suitable solvent to give N-pentyl-N-aminoguanidine hydroiodide of Formula VII. The choice of the appropriate mole ratio of the compounds of Formulae VI and III in step a) determines the completion of the reaction and the carryover of these reactants(s) into the next step and thus further also the possible formation of impurities deriving from unreacted materials deriving from unreacted materials. A mole ratio of between about 1:1.02 to 1:1.5 for the starting materials S-methylthiosemicarbazide hydroiodide and n-pentylamine, respectively, is optimal for providing the desired reaction completion with a reduced level of impurities.

The temperature of the n-pentylamine solution during the addition of S-methylthiosemicarbazide hydroiodide can be about 40° C. to 60° C., or 70° C. to 80° C., or higher.

Additionally, the choice of solvent used for the reaction can have an effect on product yield, as it has been found that lower alcohols in which the tegaserod base has a high solubility are leading to losses in yield during workup. Hence the selection of the solvent has to be done carefully to result in maximum yields.

Among the solvents that are suitable for the reaction are esters such as ethyl acetate, tertiary-butyl acetate, n-propyl acetate, isopropyl acetate, and the like, or mixtures thereof.

The mixing of the S-methylthiosemicarbazide hydroiodide and the n-pentylamine results in the formation of methylmercaptan gas. The mode of addition of the S-methylthiosemicarbazide hydroiodide to the solution of n-pentylamine in a suitable solvent can assist in controlling the generation of this gaseous material and hence the reaction conditions. More specifically, the addition of S-methylthiosemicarbazide hydroiodide to the n-pentylamine in a suitable solvent in multiple divided portions, instead of a single lot, allows a much better control over the reaction.

The reaction completion can optionally be followed by carbon treatment of the reaction mass, to remove impurities before the reaction product is progressed into the next stage, or is recovered.

The product formed in step a) can then be used in the next stage with or without isolation of the product.

Step b) involves the condensation of the product N-pentyl-N′-aminoguanidine hydroiodide of Formula VII from step a) with 5-methoxy-1H-indole-3-carboxaldehyde of Formula V in a suitable solvent and in an acidic medium. This is followed by adjusting the pH of the reaction mass to alkaline conditions to release the tegaserod base 3-(5-methoxy-1H-indol-3-ylmethylene)-N-pentylcarbazimidamide of Formula I.

Suitable temperatures for the reaction can be about 0° C. to 30° C., or higher, such as up to about 50° C.

Suitable solvents for the reaction include, without limitation thereto, tertiary butyl acetate, isopropyl acetate, acetonitrile, acetone, toluene, and water, or mixtures thereof.

An acidic medium for the reaction can be provided using inorganic acids such as, for example, hydrogen halides, sulphuric acid, nitric acid, and the like, and organic acids such as, for example, acetic acid, formic acid, and the like, or mixtures thereof. Other acids may also be used to provide an acidic medium, without limitation. The pH for conducting the reaction can range from about 0.1 to 5, or about 0.5 to 2.

Reaction completion is followed by making the pH of the reaction mass alkaline. Suitable pH ranges for the isolation of tegaserod base are about 10 to 14, or about 12 to 13. Suitable basic substances used for the pH adjustment include, but are not limited to, hydroxides, carbonates and bicarbonates of alkali metals, bases like ammonia, amines and the like.

In many instances, purification of the thus obtained compound of Formula I by slurrying, recrystallization, or a combination thereof in a suitable solvent will be desired.

Suitable solvents for slurrying or recrystallization of tegaserod base include acetonitrile, ethyl acetate, toluene, methyl ethyl ketone, diethylether, and the like or mixtures thereof.

Crystallization of the compound can be attained by forming a concentrated solution, such as at elevated temperatures, and then cooling to about 20 to 25° C. or any temperatures lower than that used to form the tegaserod solution, for example, about 0° C. to 10° C. The crystallization step may further include cooling the solution, heating the solution, or adding seed crystals to induce precipitation.

The material obtained as described above is typically further subjected to drying. Drying can be performed under reduced pressure or under atmospheric pressure at a temperature of at least about 40° C. to 90° C., or 70 to 80° C., or higher, for a sufficient time to achieve the desired residual solvent content, for example up to about 1 to 3 days. Any conventional method of drying such as, for example, vacuum tray drying, fluid bed drying, tray drying, and the like may be used and are all within the scope of this invention.

Typically, it will be desired to convert tegaserod into a salt, such as into tegaserod maleate.

Suitable solvents for the conversion of tegaserod base into tegaserod maleate include: ketones such as acetone, methyl ethyl ketone, and propanone; alcohols such as methanol, ethanol, propanol, and the like; halogenated hydrocarbons such as dichloromethane and chloroform; or mixtures thereof.

The solution of tegaserod in the solvent may be prepared by dissolution at ambient temperatures or by heating the mixture to higher temperatures, such as ranging from about 40 to 70° C.

The solution may be optionally treated with carbon for improvement of color before proceeding to addition of maleic acid for salt formation.

Small amounts of seeding crystals of the pure compound may be added to the reaction mixture. The seeding may be added before or after the addition of maleic acid to the reaction mass. Preferably, small amounts are about 1 to 20 weight %, or about 5 weight %.

The maleic acid may be added to the reaction mass directly or combined with a solvent. The solvent may be same as that used for the dissolution of tegaserod base, or may be a different solvent. Suitable solvents which can be used include ketones such as acetone, methyl ethyl ketone, and propanone; alcohols such as methanol, ethanol, propanol, and the like; halogenated hydrocarbons like dichloromethane and chloroform; or mixtures thereof.

The isolation of the compound is usually performed with stirring. The isolation step can be performed at about 20° C. to about 25° C. or at lower temperatures such as at least about 5° C.

Recovery of the tegaserod, and its maleate salt when prepared as per the above process can be performed by any means known in the art including, but not limited to filtration, centrifugation, and decanting. Tegaserod can be obtained from any composition containing a solvent or solvents which may be a suspension, solution, slurry, or an emulsion.

The obtained compound can be further dried under ambient or reduced pressure. For example, drying can be performed under reduced pressure or under atmospheric pressure at a temperature of at about 40° C. to 60° C., or about 70° C. to 80° C., or higher. Drying can be performed until the desired residual solvent content has been obtained, such as a duration of about 2 to 24 hours, or about 3 to 6 hours.

Tegaserod prepared according to this embodiment has a low level of impurities as determined by high performance liquid chromatography (“HPLC”). For example, it contains less than about 0.15 area %, or about 0.01 area %, of [(5-methoxy-1H-indol-3-ylmethylene-amino] guanidine HCl of Formula IX when measured by HPLC. 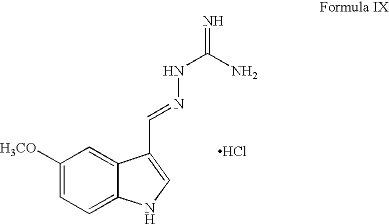
It contains less than about 0.15 area-%, or about 0.01 area-%, of 1-[(5-methoxy-1H-indol-3-yl) methylene-amino]-2-methyl isothiourea hydroiodide of Formula X when measured by HPLC.

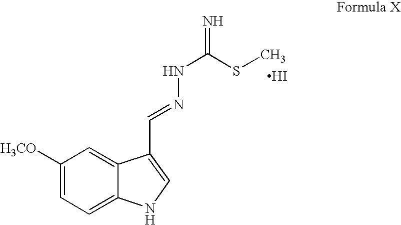

In a further aspect of the invention, there is provided a process for the preparation of the aminoguanidine impurity of tegaserod of Formula IX comprising reacting 5-methoxy-1H-indole-3-carboxaldehyde of Formula V and aminoguanidine hydrochloride of Formula II in a suitable solvent in an acidic medium. The process can be depicted as in Scheme 3.

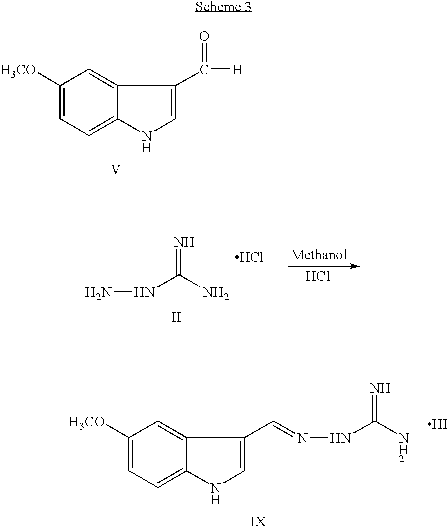

Suitable solvents for the reaction include lower alcohols like methanol, ethanol, propanol, etc, C₆-C₁₂ hydrocarbons, ethers, esters, and mixtures thereof.

Suitable strong acids which can be used for providing the acidic medium include hydrogen halides, sulphuric acid, nitric acid, and the like, and organic acids include acetic acid, formic acid, and the like, or mixtures thereof.

In yet another aspect of the invention there is provided a process for the preparation of the S-methyl impurity of Formula X comprising reacting 5-methoxy-1H-indole-3-carboxaldehyde of Formula V and S-methyl thiosemicarbazide hydroiodide of Formula VI in the presence of a suitable solvent in an acidic medium. The process can be depicted as Scheme 4.

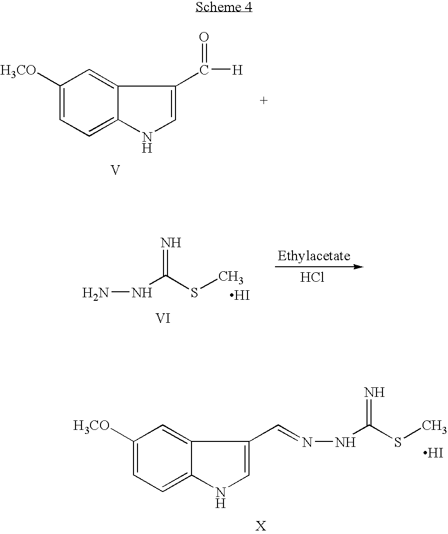

Suitable solvents for the reaction include lower alcohols like methanol, ethanol, propanol, etc, C₆-C₁₂ hydrocarbons, ethers, esters, and mixtures thereof.

Suitable strong acids which can be used for providing the acidic medium for the reaction include hydrogen halides, sulphuric acid, nitric acid, and the like, and organic acids include acetic acid, formic acid, and the like, or mixtures thereof.

Pharmaceutically acceptable salts of tegaserod include maleate, hemimaleate, oxalate, acetate, fumarate, sulfate, hydrochloride, and the like salts.

Certain aspects and embodiments of the invention are further illustrated by the following examples, which should not be construed as limiting the scope of the invention.

### EXAMPLE 1

Preparation of Tegaserod Base

Step A: Preparation of N-Pentyl-N′-Aminoguanidine Hydroiodide

31 g of n-pentylamine and 830 ml of ethyl acetate were taken into a round bottom flask and the mixture was heated to 60° C. 83 g of S-methyl thiosemicarbazide hydroiodide was added to the above reaction mass at 60° C. in 4 equal lots in about 45 minute intervals under stirring. The temperature of the reaction mass was increased to 74° C. and maintained for 2 hours. Reaction completion was checked using high performance liquid chromatography. After the reaction was completed, the temperature of the reaction mass was reduced to 30° C. Carbon was added into the reaction mass and stirred for 20 minutes. The reaction mass was then filtered and the carbon bed was washed with 350 ml of ethyl acetate.

Step B: Preparation of Tegaserod Base

The combined ethyl acetate filtrate from Step A was taken and 50 g of 5-methoxy-1H-indole-3-carboxaldehyde was added to it. The pH of the reaction mass was made acidic using hydrochloric acid. After the reaction mass pH of 0.7 was achieved, the reaction mass was maintained at 30 to 35° C. for 1 hour. Reaction completion was checked using thin layer chromatography. After the reaction was completed, the reaction mass was filtered and the solid was washed with 150 ml of ethyl acetate. The wet compound was again slurried in 1000 ml of ethyl acetate for 1 hour. The solid was then filtered and washed with 150 ml of ethyl acetate. The above wet compound was taken into a fresh round bottom flask and 2000 ml of water was added to it. The pH of the mixture was adjusted to 12 to 12.5 using aqueous sodium hydroxide solution under stirring. After the pH adjustment was completed, the reaction mass was stirred for 1 hour. The reaction mass was then filtered and the solid was washed with 300 ml of water. The solid was dried under vacuum. The wet solid was again slurried in 300 ml of water and then filtered and washed with 2000 ml of water. The wet solid was initially dried under vacuum followed by drying in air oven at a temperature of 55 to 60° C. for 11 hours to give 74.9 g of the title compound.

Step C: Purification of Tegaserod Base

71 g of the tegaserod obtained above was taken into a round bottom flask and 300 ml of acetonitrile were added to it. The reaction mass was heated to 78° C. and maintained for 35 minutes. The reaction mass was checked for clear dissolution. After clear dissolution was obtained, the temperature of the reaction mass was reduced to 25 to 30° C. The reaction mass was maintained at 25 to 30° C. for 1 hour. The reaction mass was then filtered and washed with 75 ml of acetonitrile. The wet compound was initially suction dried under vacuum followed by drying in air oven at a temperature of 60° C. for 4 to 5 hours to yield 61.2 g of the title compound.

### EXAMPLE 2

Preparation of Tegaserod Maleate:

25 g of pure tegaserod base and 750 ml of ethyl methyl ketone was taken into a round bottom flask and the mixture was stirred at 28° C. for 10 minutes. The mixture was checked for clear dissolution. Carbon was added to the reaction mass and stirred for 20 minutes. The reaction mass was then filtered and the carbon bed was washed with 50 ml of ethyl methyl ketone. The combined filtrates were taken into a round bottom flask and stirred. Seed crystals of the maleate salt was added. A particle free solution of 10.6 g maleic acid in 125 ml of ethyl methyl ketone was added slowly at 28° C. The reaction mass was then filtered and the solid was washed with 50 ml of ethyl methyl ketone. The obtained compound was dried initially at 30° C. for 3.5 hours followed by drying at 60° C. for 10 hours to get 30.8 g of the title compound.

### EXAMPLE 3

Preparation of Aminoguanidine Impurity (Formula IX)

20 g of 5-methoxy-1H-indole-3-carboxyaldehyde of Formula IV, 400 ml of methanol and 13 g of aminoguanidine hydrochloride were taken into a round bottom flask and stirred for about 5 minutes with a simultaneous adjustment of pH with 0.4 ml of HCl to about 3.4. The reaction mass was maintained for 2.5 hours at 28° C. and then 80% of the solvent was distilled from the reaction mass at 47° C. The reaction mass was then cooled to 0° C. under stirring. The reaction mass was maintained at 0° C. for 30 minutes. The solid obtained was filtered and washed with 40 ml of methanol followed by drying at 28° C. under vacuum for 5 hours to yield 22.5 g of the title compound.

### EXAMPLE 4

Preparation of S-Methyl Impurity (Formula X)

100 ml of ethyl acetate and 6.65 g of S-methyl thiosemicarbazide hydroiodide were taken into a round bottom flask and stirred for about 10 minutes followed by the addition of 5 g of 5-methoxy-1H-indole-3-carboxyaldehyde with further continuation of the stirring. The pH of the reaction mass was adjusted to 0.4 with 0.3 ml of hydrochloric acid and the reaction mass was maintained for about 55 minutes at 28° C. until the solid separated out. The solid obtained was filtered and washed with ethyl acetate, then was dried at 60° C. for 6 hours. The dried compound was taken into 100 ml of water, and the pH was adjusted to 11.5 under stirring with 20% aqueous sodium hydroxide solution. After the pH adjustment was complete, the mass was maintained under stirring for 20 minutes. The separated solid was filtered and washed with 25 ml of water. The compound was dried in air oven at 60° C. for 3 hours to obtain 4.5 g of the title compound.

We claim:

1. A process for preparing tegaserod, comprising reacting S-methylthiosemicarbazide hydroiodide with n-pentylamine to form N-pentyl-N-aminoguanidine hydroiodide, in an ester solvent.

2. The process of claim 1, wherein an ester solvent comprises one or more of ethyl acetate, tertiary-butyl acetate, and propyl acetate.

3. The process of claim 1, wherein an ester solvent comprises ethyl acetate.

4. The process of claim 1, wherein a mole ratio of S-methylthiosemicarbazide hydroiodide to n-pentylamine is about 1:1.02 to 1:1.5, respectively.

5. The process of claim 1, wherein S-methylthiosemicarbazide hydroiodide is added to n-pentylamine in multiple divided portions.

6. The process of claim 1, wherein N-pentyl-N-aminoguanidine hydroiodide is condensed with 5-methoxy-1H-indole-3-carboxaldehyde in an acidic medium, to form tegaserod.

7. The process of claim 6, wherein N-pentyl-N-aminoguanidine hydroiodide is formed in an ester solvent, and condensed without isolation.

8. The process of claim 6, wherein an acidic medium has a pH about 0.1 to about 5.

9. The process of claim 6, wherein an acidic medium has a pH about 0.5 to about 2.

10. The process of claim 6, wherein formed tegaserod is separated by adjusting pH of a medium to an alkaline value.

11. The process of claim 6, wherein formed tegaserod is separated by adjusting pH of a medium to about 10 to about 14.

12. The process of claim 6, wherein formed tegaserod is separated by adjusting pH of a medium to about 12 to about 13.

13. The process of claim 6, wherein tegaserod is separated and purified by slurrying or crystallization.

14. The process of claim 6, wherein tegaserod is converted to a salt.

15. A process for preparing tegaserod, comprising:
reacting S-methylthiosemicarbazide hydroiodide with n-pentylamine to form N-pentyl-N-aminoguanidine hydroiodide, in an ester solvent; and
condensing N-pentyl-N-aminoguanidine hydroiodide with 5-methoxy-1H-indole-3-carboxaldehyde in an acidic medium, to form tegaserod.

16. The process of claim 15, wherein N-pentyl-N-aminoguanidine hydroiodide is not isolated from an ester solvent, before condensing.

17. The process of claim 15, wherein an ester solvent comprises ethyl acetate.

18. The process of claim 15, wherein a mole ratio of S-methylthiosemicarbazide hydroiodide to n-pentylamine is about 1:1.02 to 1:1.5, respectively.

19. The process of claim 15, wherein S-methylthiosemicarbazide hydroiodide is added to n-pentylamine in multiple divided portions.

20. The process of claim 15, wherein an acidic medium has a pH about 0.5 to about 2.
